(19) United States
(12) Patent Application Publication
(10) Pub. No.: US 2023/0372112 A1
(43) Pub. Date: Nov. 23, 2023

(54) SUBTALAR JOINT ARTHROPLASTY

(71) Applicant: New York Society for the Relief of the Ruptured and Crippled, Maintaining the Hospital for Special S, New York, NY (US)

(72) Inventors: Daniel R. Sturnick, Geneseo, NY (US); Constantine A. Demetracopoulos, North Salem, NY (US)

(21) Appl. No.: 18/248,110

(22) PCT Filed: Oct. 1, 2021
(86) PCT No.: PCT/US2021/053147

§ 371 Date: Apr. 6, 2023

Related U.S. Application Data
(60) Provisional application No. 63/104,406, filed on Oct. 22, 2020.

(51) Int. Cl. A61F 2/42; A61F 2/46; A61B 17/17; B33Y 80/00

(52) U.S. Cl. CPC A61F 2/4202; A61F 2/4606; A61B 17/1775; B33Y 80/00; A61F 2002/4207; A61F 2002/302

(57) ABSTRACT

A subtalar joint replacement includes a talar component and a calcaneal component. The talar component includes a talar fixation surface, a talar articular surface opposite the talar fixation surface, the talar articular surface having a first shape, and one or more fixation devices extending from the talar fixation surface. The calcaneal component includes a calcaneal fixation surface, a calcaneal articular surface opposite the calcaneal fixation surface, the calcaneal articular surface having a second shape that interfaces with the first shape of the talar articular surface to generate constraint across a subtalar joint, and one or more fixation devices extending from the calcaneal fixation surface.

### TECHNICAL FIELD

This disclosure relates to orthopedic systems, methods, and devices.

### BACKGROUND

Pathology of the subtalar joint is high in its incidence and often occurs concomitant to ankle arthritis, as well as with other conditions, such as flatfoot deformity. Total ankle replacement (TAA) is often performed in patients with ankle arthritis and/or pathology of the subtalar joint in order to limit further subtalar joint degeneration and relieve pain.

In addition, for severe cases of subtalar joint degeneration, a subtalar joint arthrodesis is often performed in conjunction with TAA. Subtalar joint arthrodesis is utilized in a wide range of pathologies, including primary and posttraumatic arthritis, comminuted calcaneal fractures, talocalcaneal coalitions, and posterior tibial tendon dysfunction. However, TAA procedures and subtalar joint arthrodesis are associated with numerous complications, such as non-union, screw trajectory, and joint degeneration. In addition, the range of motion of the hindfoot and ankle joint is often substantially reduced following subtalar joint arthrodesis.

### SUMMARY

In one aspect, a subtalar joint replacement includes a talar component and a calcaneal component. The talar component includes a talar fixation surface, a talar articular surface opposite the talar fixation surface, the talar articular surface having a first shape, and one or more fixation devices extending from the talar fixation surface. The calcaneal component includes a calcaneal fixation surface, a calcaneal articular surface opposite the calcaneal fixation surface, the calcaneal articular surface having a second shape that interfaces with the first shape of the talar articular surface to generate constraint across a subtalar joint, and one or more fixation devices extending from the calcaneal fixation surface.

Embodiments can include one or more of the following features in any combination.

In certain embodiments, the first shape of the talar articular surface conforms to a surface of a portion of a first torus, and the second shape of the calcaneal articular surface conforms to a surface of a portion of a second torus

In some embodiments, the first torus has a first articular radius, and the second torus has a second articular radius different from the first articular radius.

In certain embodiments, the talar fixation surface is shaped to conform to native anatomy of an articular surface a talus bone, and the calcaneal fixation surface is shaped to conform to native anatomy of an articular surface a calcaneus bone.

In some embodiments, the talar fixation surface is shaped to conform to a surface of a section of a first cone, and the calcaneal fixation surface is shaped to conform to a surface of a section of a second cone.

In certain embodiments, at least one of the talar component and the calcaneal component has a non-uniform thickness.

In some embodiments, at least one of the talar component and the calcaneal component has a thickness that increases in a lateral direction and in an anterior direction.

In certain embodiments, the subtalar joint replacement includes a bearing insert configured to be positioned between the talar component and the calcaneal component.

In some embodiments, the bearing insert is affixed to the talar articular surface or the calcaneal articular surface.

In certain embodiments, the subtalar joint replacement includes a rail formed on the talar articular surface and the calcaneal articular surface, and a slot formed on at least one surface of the bearing insert, the slot configured to slidably engage the rail.

In another aspect, a system includes a subtalar joint replacement device, a set of talar cutting guides, a set of calcaneal cutting guides, and a surface prep tool. The subtalar joint replacement device includes a talar component and a calcaneal component.

Embodiments can include one or more of the following features in any combination.

In some embodiments, the set of talar cutting guides includes a first talar cutting guide with a first set of openings extending along a curved length of the first talar cutting guide, and a second talar cutting guide with a second set of openings extending along a curved length of the second talar cutting guide, the second set of openings being offset from the first set of openings; and the set of calcaneal cutting guides includes a first calcaneal cutting guide with a third set of openings extending along a curved length of the first calcaneal cutting guide, and a second calcaneal cutting guide with a fourth set of openings extending along a curved length of the second calcaneal cutting guide, the fourth set of openings being offset from the third set of openings.

In certain embodiments, the system includes an alignment tool configured to indicate an orientation of a subtalar joint and a size of the subtalar joint.

In some embodiments, the alignment tool incudes a handle, and a tool end coupled to the handle, the tool end including two or more guide holes.

In certain embodiments, the tool end of the alignment tool includes one or more radiopaque markers.

In some embodiments, the set of talar cutting guides includes a first and second patient-specific talar cutting guide each having a surface that conforms to a surface of a talar bone of a patient, and the set of calcaneal cutting guides includes a first and second patient-specific calcaneal cutting guides each having a surface that conforms to a surface of a calcaneal bone of the patient.

In certain embodiments, the surface prep tool includes a handle, and a tool end coupled to the handle. The tool end includes one or more holes, and a roughened surface.

In some embodiments, the system includes a bearing insert configured to be positioned between the talar component and the calcaneal component.

In certain embodiments, the talar component includes a talar fixation surface, a talar articular surface opposite the talar fixation surface, the talar articular surface having a first shape; and the calcaneal component includes a calcaneal fixation surface, and a calcaneal articular surface opposite the calcaneal fixation surface, the calcaneal articular surface having a second shape that interfaces with the first shape of the talar articular surface to generate constraint across a subtalar joint.

In another aspect, a method includes determining an orientation and a size of a subtalar joint of a patient; forming a curved resected surface on an articular surface of a talus bone of the patient using a first pair of cutting guides, the curved resected surface having a shape that conforms to native anatomy of the articular surface the talus bone; forming a curved resected surface on an articular surface of a calcaneus bone of the patient using a second pair of cutting guides, the curved resected surface having a shape that conforms to native anatomy of the articular surface the calcaneus bone; coupling a talar component of a subtalar joint replacement device to the talus bone, the talar component having a fixation surface corresponding to the shape of the curved resected surface on the articular surface of the talus bone; and coupling a calcaneal component of the subtalar joint replacement device to the calcaneus bone, the calcaneal component having a fixation surface corresponding to the shape of the curved resected surface on the articular surface of the calcaneus bone.

Advantages of the systems, devices, and methods described herein can include preservation of the natural, anatomic kinematics of the subtalar joint, as well as enable many other joints in the foot and ankle to maintain function following treatment of subtalar joint arthritis. The systems, devices, and methods described herein can also enable intra-articular deformity correction and improve hindfoot alignment at the subtalar joint. The systems, devices, and methods described herein can provide improved load transfer through subtalar joint. The systems, devices, and methods described herein can provide different levels of constraint across the subtalar joint by utilizing different shapes and conformity of implants, which allows for improved correction of particular conditions, such as flatfoot conditions and stiff post-traumatic arthritis. The systems, devices, and methods described herein can also enable shaping of conical bone cuts to more closely mimic the anatomical surfaces of the subtalar joint, thus reducing the amount of bone that must be resected to prepare the joint for an implant. The systems, devices, and methods described herein can provided improved fixation stability of subtalar joint implants through improved fixation surface preparation techniques. The systems, devices, and methods described herein can also enable improved patient outcomes for subtalar joint arthritis treatment due to patient-specific implants and resection guides. The systems, devices, and methods described herein can complement and protect other procedures performed proximate to the subtalar joint, such as soft tissue reconstructions and osteotomies. The systems, devices, and methods described herein can enable reduced procedure and recovery times. In addition, the systems, devices, and methods described herein can provide a reduced learning curve across a wide range of surgeon skill-levels The systems, devices, and methods described herein can help preserve motion of the adjacent joints (e.g., compared to arthrodesis). The systems, devices, and methods described herein can improve longevity of ankle replacements. The systems, devices, and methods described herein can help correct deformities in the calcaneus, talus, and hindfoot joint, such as coronal, Sagittal and axial plane deformities, including Valgus hindfoot, subtalar deformity, and volar angle deformity.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1 depicts a subtalar joint replacement device implanted in a subtalar joint of a patient.

FIG. 2 depicts a perspective view of a talar component of the subtalar joint replacement device of FIG. 1.

FIG. 3 depicts a perspective view of a calcaneal component of the subtalar joint replacement device of FIG. 1.

FIG. 4A depicts the articular surface of a talus bone.

FIG. 4B depicts the articular surface of a calcaneus bone.

FIG. 5 depicts a talar component of the subtalar joint replacement device of FIG.

FIG. 6 depicts a calcaneal component of the subtalar joint replacement device of FIG. 1.

FIG. 7 depicts a talar component of the subtalar joint replacement device of FIG.

FIG. 8 depicts a calcaneal component of the subtalar joint replacement device of FIG. 1.

FIG. 9 depicts a perspective view of a talar component having a non-uniform thickness.

FIG. 10 depicts a perspective view of a talar component and bearing insert of the subtalar joint replacement device of FIG. 1.

FIG. 11 depicts a perspective view of a calcaneal component and bearing insert of the subtalar joint replacement device of FIG. 1.

FIG. 12 depicts a perspective view of a mobile bearing insert and corresponding calcaneal component.

FIG. 13A depicts a perspective view of the mobile bearing insert of FIG. 12.

FIG. 13B depicts a perspective view of the calcaneal component of FIG. 12.

FIG. 14 depicts a perspective view of a bearing insert with a non-uniform thickness.

FIG. 15 depicts another subtalar joint replacement device implanted in a subtalar joint of a patient.

FIG. 16 depicts a perspective view of an alignment tool for use in implanting a subtalar joint replacement device.

FIGS. 17A and 17B depict front views of talar cutting guides for use in implanting a subtalar joint replacement device.

FIGS. 18A and 18B depict front views of calcaneal cutting guides for use in implanting a subtalar joint replacement device.

FIG. 19 depicts a perspective view of a patient-specific cutting guide for use in implanting a subtalar joint replacement device.

FIG. 20 depicts a perspective view of a surface prep tool for use in implanting a subtalar joint replacement device.

FIGS. 21-28 depict an exemplary method of implanting the subtalar joint replacement device of FIG. 1.

Like reference symbols in the various drawings indicate like elements.

### DETAILED DESCRIPTION

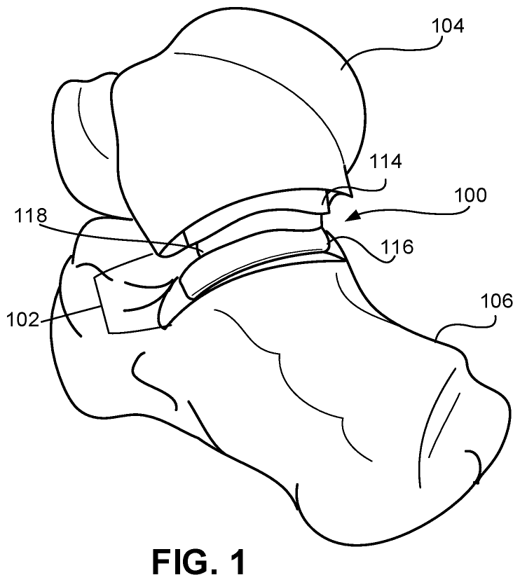

Referring to FIG. 1, a subtalar joint replacement device 100 is depicted as being implanted within the subtalar joint 102 between the talus bone 104 and calcaneus bone 106. As depicted in FIG. 1, the subtalar joint replacement device 100 includes a talar component 114 configured to be attached to the talus bone 104 and a calcaneal component 116 configured to be attached to the calcaneus bone 106. In addition, as depicted in FIG. 1, the subtalar joint replacement device 100 includes a bearing insert 118 positioned between and contacting the talar component 114 and calcaneal component 116.

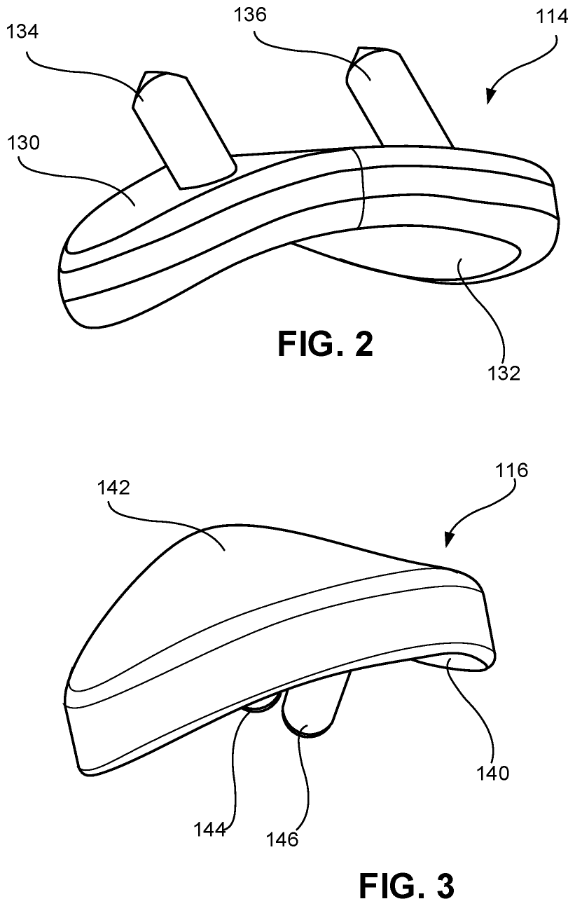

FIG. 2 depicts the talar component 114 of the subtalar joint replacement device 100 of FIG. 1. As can be seen in FIG. 2, the talar component 114 has a fixation surface 130 and an articular surface 132 opposite the fixation surface 130. The fixation surface 130 of the talar component 114 has a generally convex shape and is configured to contact and interface with articular surface the talus bone 104 of a patient. The articular surface 132 of the talar component 114 has a generally concave shape and is configured to contact and interface with a bearing element (such as bearing insert 118 of FIG. 1). As will be described in further detail herein, the shape of the fixation surface 130 and the shape of the articular surface 132 can each be configured to provide a particular level of constraint and stability across the subtalar joint 102.

The talar component 114 can be formed of any suitable material, such as metal, plastic, or ceramic. For example, in some implementations, the talar component 114 is composed of titanium or cobalt-chrome alloys. In some implementations, the talar component 114 is formed using additive manufacturing, a molding process, or an extrusion process. For example, the talar component 114 can be composed of titanium and the articular surface of the talar component 114 can be treated with a material coating, such as a ceramic coating, for improved wear properties when interfacing a polyethylene material forming the bearing insert 118.

As can be seen in FIG. 2, the talar component 114 includes two fixation devices 134, 136. The fixation devices 134, 136 of the talar component 114 are configured to be inserted into openings formed in the talus bone 104 (e.g., pilot holes drilled into the talus bone 104) and attach the talar component 114 of the subtalar joint replacement device 100 to the talus bone 104. For example, the fixation devices 134, 136 are configured to attach the talar component 114 to the talus bone 104 such that the fixation surface 130 of the talar component 114 is flush with the resected surface of the talus bone 104. In some implementations, the fixation devices 134, 136 are impacted into holes within the talus bone 104 to couple the talar component 114 to the talus bone 104.

The fixation devices 134, 136 of the talar component 114 can be any suitable fixation devices including, but not limited to, pins, pegs, stems, posts, keels, etc. The fixation devices 134, 136 of the talar component 114 can be made of any suitable material, such as porous metal (such as a porous titanium alloy) for bone ingrowth. In some implementations, the fixation devices 134, 136 are extruded onto the fixation surface 130 of the talar component 114. In some implementations, the fixation devices 134, 136 are formed using a combination of molding, extrusion, and/or machining. In some implementations, the fixation devices 134, 136 are formed on the talar component 114 using 3D printing. Other implementations may include fixation devices 134, 136 with modular attachment to the talar component 114 body. In addition, while FIG. 2 depicts the talar component 114 as having two fixation devices 134, 136, any suitable number of fixation devices can be used. For example, in some implementations, the talar component 114 includes a single fixation device for attaching the talar component 114 to the talus bone 104. In some implementations, the talar component 114 includes three or more fixation devices for attaching the talar component 114 to the talus bone 104.

FIG. 3 depicts the calcaneal component 116 of the subtalar joint replacement device 100 of FIG. 1. As can be seen in FIG. 3, the calcaneal component 116 has a fixation surface 140 and an articular surface 142 opposite the fixation surface 140. The fixation surface 140 of the calcaneal component 116 has a generally concave shape and is configured to contact and interface with the articular surface of the calcaneus bone 106 of a patient. The articular surface 142 of the calcaneal component 116 has a generally convex shape and is configured to contact and interface with a bearing element of the device 100 (such as bearing insert 118 of FIG. 1). As will be described in further detail herein, the shape of the fixation surface 140 and the shape of the articular surface 142 can each be configured to provide a particular level of constraint and stability across the subtalar joint 102.

The calcaneal component 116 can be formed of any suitable material, such as metal, plastic, or ceramic. For example, in some implementations, the calcaneal component 116 is composed of titanium or cobalt chrome alloys. In some implementations, the calcaneal component 116 is formed using a molding process, an extrusion process, or additive manufacturing.

As can be seen in FIG. 3, the calcaneal component 116 includes two fixation devices 144, 146. The fixation devices 144, 146 of the calcaneal component 116 are configured to be inserted into openings formed in the calcaneus bone 106 (e.g., pilot holes drilled into the calcaneus bone 106), and to attach the calcaneal component 116 of the subtalar joint replacement device 100 to the calcaneus bone 106. The fixation devices 144, 146 are configured to attach the calcaneal component 116 to the calcaneus bone 106 such that the fixation surface 140 of the calcaneal component 116 is flush with the resected surface of the calcaneus bone 106. In some implementations, the fixation devices 144, 146 are impacted into holes within the calcaneus bone 106 to couple the calcaneal component 114 to the calcaneus bone 106.

The fixation devices 144, 146 of the calcaneal component 116 can be any suitable fixation devices including, but not limited to, pins, pegs, stems, posts, keels, etc. The fixation devices 144, 146 of the calcaneal component 116 can be made of any suitable material, such as porous metal (such as a porous titanium alloy). In some implementations, the fixation devices 144, 146 are extruded onto the fixation surface 140 of the calcaneal component 116. In some implementations, the fixation devices 144, 146 are formed using a combination of molding, extrusion, and/or machining. In some implementations, the fixation devices 144, 146 are formed on the calcaneal component 116 using 3D printing. Other implementations may include fixation devices 144, 146 with modular attachment to the calcaneal component 116 body

In addition, while FIG. 3 depicts the calcaneal component 116 as having two fixation devices 144, 146, any suitable number of fixation devices can be used. For example, in some implementations, the calcaneal component 116 includes a single fixation device for attaching the calcaneal component 116 to the calcaneus bone 106. In some implementations, the calcaneal component 116 includes three or more fixation devices for attaching the calcaneal component 116 to the calcaneus bone 106.

As previously mentioned, the shape of the fixation surfaces 130, 140 and the shape of the articular surfaces 132, 142 of talar component 114 and the calcaneal component 116 can be altered to provided different amounts of constraint across the subtalar joint 102.

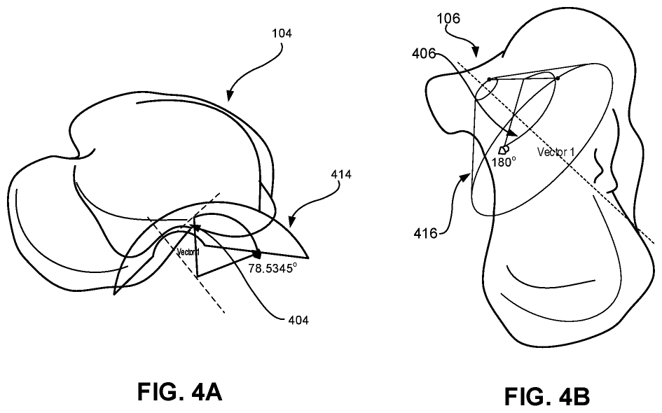

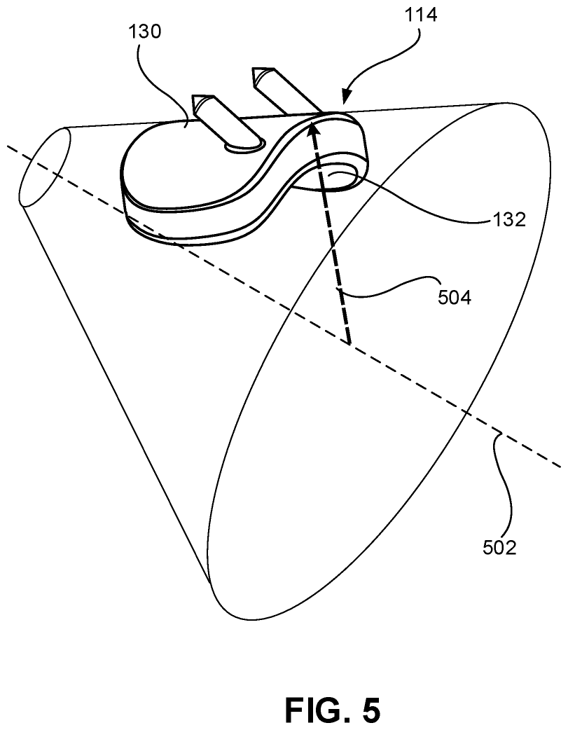

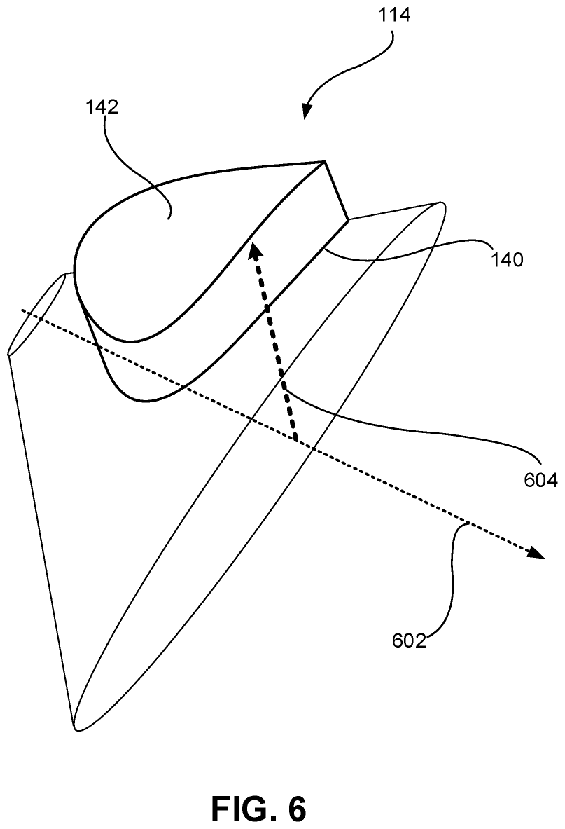

As depicted in FIGS. 4A, the native articular surface 404 of the talus bone 104 conforms to the surface of a section of a cone 414. As such, in some implementations, the fixation surface 130 of the talar component 114 is shaped such that the fixation surface 130 conforms to the surface of a section of a cone in order to mimic the shape of the native anatomy of the articular surface 404 of the talus bone 104. For example, as depicted in FIG. 2, the fixation surface 130 of the talar component 114 is defined by a taper in the anteromedial direction, giving the fixation surface 130 a shape that conforms to the surface of a cone section. As depicted in FIG. 5, the fixation surface 130 of the talar component 114 extends about a cone axis 502 and has a fixed distance relative to the cone axis 502 in the radial direction 504. Several benefits are provided by utilizing a fixation surface that with a shape that mimics the native anatomy of the articular surface 404 of the talus bone 104, including increased fixation surface area, improved constraint of fixation, and minimized bone resection.

As depicted in FIG. 4B, the native articular surface 406 of the calcaneus bone 106 also has a shape that conforms to the surface of a section of a cone 416. In some implementations, the fixation surface 140 of the calcaneal component 116 is shaped such that the fixation surface 130 conforms to the surface of a cone section, which mimics the shape of the native anatomy of the articular surface 406 of the calcaneus bone 106. For example, as depicted in FIG. 3, the fixation surface 140 of the calcaneal component 116 is defined by a taper in the anteromedial direction, giving the fixation surface 140 of the calcaneal component 116 a shape that conforms to the surface of a section of a cone. Further, as depicted in FIG. 6, the fixation surface 140 of the calcaneal component 116 extends about a cone axis 602 and has a fixed distance relative to the cone axis 602 in the radial direction 604. Several benefits are provided by utilizing a fixation surface with a shape that mimics the native anatomy of the articular surface 406 of the calcaneus bone 106, including increased fixation surface area, improved constraint of fixation, and minimized bone resection.

The fixation surface 130, 140 of both the talar component 114 and the calcaneal component 116 have been depicted as being shaped to conform to the surface of a section of a cone, which mimics native anatomy and provide high levels of fixation stability. However, the fixation surface 130 of the talar component 114 and the fixation surface 116 of the calcaneal component 116 can have other surface shapes. For instance, in some implementations, the fixation surfaces 130, 140 of one or more of the talar and calcaneal components 114, 116 is shaped to conform the surface of a portion of a torus to provide added constraint across the subtalar joint 102. In some implementation, the fixation surfaces 130, 140 of one or more of the talar and calcaneal components 114, 116 are shaped to conform to the surface of a portion of a cylinder. Other possible shapes for the fixation surfaces 130, 140 of one or more of the talar and calcaneal components 114, 116 can include a flat surface, spherical surface, or a saddle shaped surface. The shape of the fixation surfaces 130, 140 of one or more of the talar and calcaneal components 114, 116 can be selected based on the shape of the respective bones 104, 106 to which the components are fixed.

In addition, while the fixation surface 130, 1340 of both the talar component 114 and the calcaneal component 116 have been depicted as having the same shape, in some implementations the fixation surface 130 of the talar component 114 and the fixation surface 140 of the calcaneal component 116 can each have different shapes. For example, the fixation surface 130 of the talar component 114 can be shaped to conform to the surface of a portion of a torus while the fixation surface 140 of the calcaneal component 116 can be shaped to conform to the surface of a cone section, and vice versa.

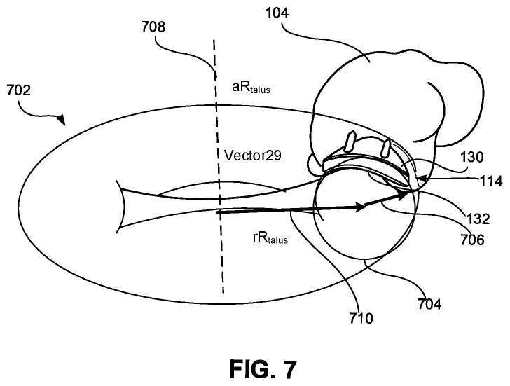

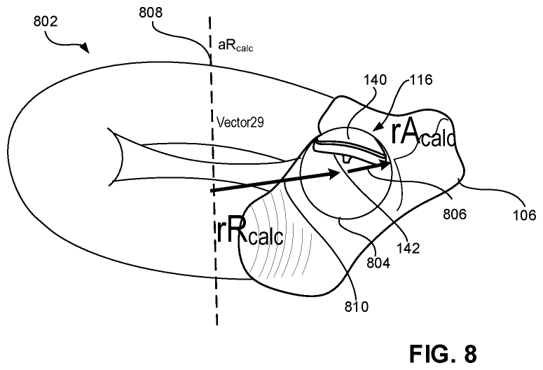

In addition to altering the shape of the fixation surface 130, 140 of one or both of the talar component 114 and the calcaneal component 116, the shape of the articular surface 132, 142 of each component 114, 116 can be designed to provide particular levels of constraint across the subtalar joint 102. For example, as depicted in FIGS. 2 and 7, in some implementations, the articular surface 132 of the talar component 114 is shaped to conform to the surface of a portion of a torus 702. Similarly, as depicted in FIGS. 3 and 8, in some implementations, the articular surface 142 of the calcaneal component 116 is shaped to conform to the surface of a portion of a torus 802.

As previously discussed and depicted in FIGS. 4A and 4B, the articular surface 404 of the talus bone 104 and the articular surface 406 of the calcaneus bone 106 are each shaped to conform to the surface of a section of a cone 414, 416. As such, by designing the articular surface 132, 142 of one or both of the talar component 114 and calcaneal component 116 to conform to the surface of a portion of a torus, the constraint across the subtalar joint 102 can be increased compared to a device with articular surfaces that conform to the native anatomy. By increasing the level of constraint across the subtalar joint 102 through torus-conforming articular surfaces 132, 142, the range of motion across the subtalar joint 102 is reduced, which increases the stability of the subtalar joint 102 following implantation of the subtalar joint replacement device 100. As such, a subtalar joint replacement device 100 with talar and calcaneal components 104, 106 with articular surfaces 132, 142 conforming to a surface of a portion of a torus may provide improved clinical outcomes by providing increased stability across the subtalar joint 102 while still allowing for normal motion of the subtalar joint 102.

As depicted in FIGS. 7, the torus 702 to which the articular surface 132 of the talar component 114 conforms has a circular profile 704 defined by a particular articular radius 706 (rAₜₐₗᵤₛ). The circular profile 704 is revolved around a central torus axis 708 (aRₜₐₗᵤₛ) that is located a fixed distance away from the center of the circular profile 704 in order to form a rotational radius 710 (rRₜₐₗᵤₛ) of the torus 702.

Similarly, as depicted in FIG. 8, the torus 802 to which the articular surface 142 of the calcaneal component 116 conforms has a circular profile 804 defined by a particular articular radius 806 (rA_{calc}). The circular profile 804 is revolved around a central torus axis 808 (aR_{calc}) that is located a fixed distance away from the center of the circular profile 804 in order to form a rotational radius 810 (rR_{calc}) of the torus 802.

The articular radii 706, 806 and the rotational radii 710, 810 of the toruses 702, 802 used to form the articular surface 132, 142 of the talar component 114 and calcaneal component 116, respectively, can be adjusted to provide various levels of constraint across the subtalar joint 102 resulting from the interaction of the articular surfaces 132, 142. For example, the closer the articular radius 706 of the torus 702 used to define the articular surface 132 of the talar component 114 is to the articular radius 806 of the torus 802 used to define the articular surface 142 of the calcaneal component 116, the more constraint that is provided across the subtalar joint 102 due to interaction of the articular surfaces 132, 142 of the subtalar joint replacement device 100. Conversely, the greater the difference between the articular radius 706 of the torus 702 used to define the articular surface 132 of the talar component 114 and the articular radius 806 of the torus 802 used to define the articular surface 142 of the calcaneal component 116 (e.g., rA_{calc}>rAₜₐₗᵤₛ or rA_{calc}<rAₜₐₗᵤₛ), the less constraint that the subtalar joint replacement device 100 will provide across the subtalar joint 102. In some implementations, the size of each of the talar and calcaneal components 104, 106 are selected on a patient-specific basis to optimize the stability and freedom-of-motion across the subtalar joint 102 of the patient.

While the articular surface 132, 142 of both the talar component 114 and the calcaneal component 116 have been depicted as being shaped to conform to the surface of a portion of a torus, the articular surface 132 of the talar component 114 and/or the articular surface 142 of the calcaneal component 116 can have other surface shapes. For instance, in some implementations, the articular surface 132, 142 of one or more of the talar and calcaneal components 114, 116 is shaped to conform to the surface of a section of a cone in order to mimic the native anatomy of the subtalar joint 102, which reduces constraint and stability across the subtalar joint 102. Further, if the articular surfaces 132, 142 of the talar and calcaneal components 114, 116 are shaped to conform to the surface of a section of a cone, the relative value of the conical axis used to form the articular surface 132 of the talar component 114 compared to of the conical axis used to form the articular surface 142 of the calcaneal component 116 can be adjusted to provide increased or decreased constraint between the articular surfaces 132, 142 and across the subtalar joint 102. In some implementation, the articular surfaces 132, 142 of one or more of the talar and calcaneal components 114, 116 are shaped to conform to the surface of a portion of a cylinder. In some implementations, using a subtalar joint replacement device 100 with talar and calcaneal components 114, 116 having articular surfaces 132, 142 shaped to conform to the surface of a portion of a cylinder increases constraint and stability across the subtalar joint 102. Other possible shapes for the articular surfaces 132, 142 of one or more of the talar and calcaneal components 114, 116 can include a flat surface, spherical surface, or a saddle shaped surface. The shape of the articular surfaces 132, 142 of one or more of the talar and calcaneal components 114, 116 can be selected based on the articular constraints desired across the subtalar joint 102.

In addition, while the articular surface 132, 142 of both the talar component 114 and the calcaneal component 116 have been depicted as having the same shape, in some implementations the articular surface 132 of the talar component 114 and the articular surface 142 of the calcaneal component 116 can each have different shapes. For example, the articular surface 132 of the talar component 114 can be shaped to conform to the surface of a portion of a torus while the articular surface 142 of the calcaneal component 116 can be shaped to conform to the surface of a cone section, and vice versa.

In addition to altering the shape of the fixation surfaces 130, 140 and the shape of the articular surfaces 132, 142 of the talar component 114 and calcaneal component 116, the thickness of each of the components 114, 116 of the subtalar joint replacement device 100 can be adjusted to correct one or more deformities of the subtalar joint 102, such as flatfoot and hindfoot deformities. For example, in cases in which the subtalar joint 102 does not present any deformities, talar and calcaneal components with a uniform thickness (e.g., talar component 114 and calcaneal component 116 in FIGS. 1-3) can be used in the subtalar joint replacement device 100. However, if the subtalar joint 102 has one or more deformities, the thickness of the talar component 114 and/or of the calcaneal component 116 can be non-uniform (variable) to compensate for and correct the joint deformities.

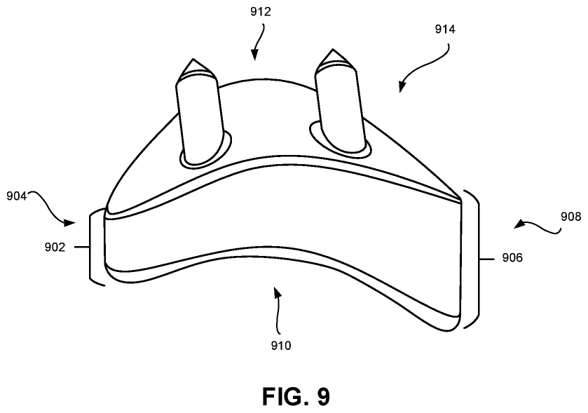

For example, FIG. 9 depicts a talar component 914 with a non-uniform thickness that can be used to correct deformities in the subtalar joint 102 and improve alignment across the subtalar joint 102. As can be seen in FIG. 9, the talar component 914 has a first thickness 902 at the medial side 904 of the talar component 914 and a second, larger thickness 906 at the lateral side 908 of the talar component 914, with the thickness of the talar component 914 gradually increasing from the medial side 904 to the lateral side 908. In addition, the thickness of the talar component 914 gradually increases from the posterior side 910 to the anterior side 912. Thus, the thickness of the talar component 914 increases in both the anterior direction and the lateral direction, which can promote improved varus component alignment in the case of valgus hindfoot deformity. In some implementations, the thickness of the talar components can increase in the medial direction and/or in the posterior direction.

In some embodiments, the thickness of the calcaneal component 116 is alternatively or additionally non-uniform to correct deformities of the subtalar joint 102. For example, the thickness of the calcaneal component 116 can be non-uniform in the medial-lateral direction and/or in the anterior-posterior direction. By varying the thickness of the talar component 114 and/or the thickness of the calcaneal component 116 in one or more directions, multiplanar deformities within the subtalar joint 102 can be corrected, and alignment across the subtalar joint 102 can be improved.

In some implementations, the thickness of the talar component 114 and/or calcaneal component 116 may be increased or decreased to accommodate different amounts of resection of the talus bone 104 and calcaneus bone 106, respectively. For example, if larger amounts of the talus bone 104 must be resected, for example due to the presence diseased bone or other complications, a talar component 114 with an increased thickness can be used to compensate for the increased amount of resected bone 104.

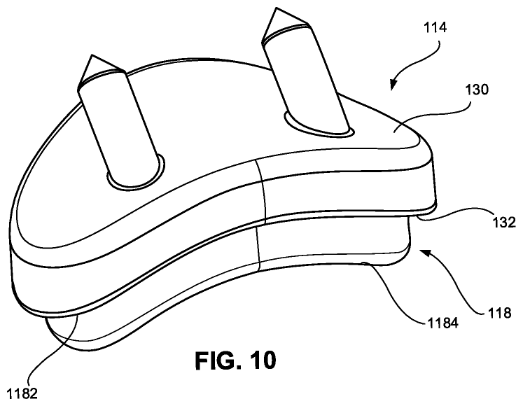

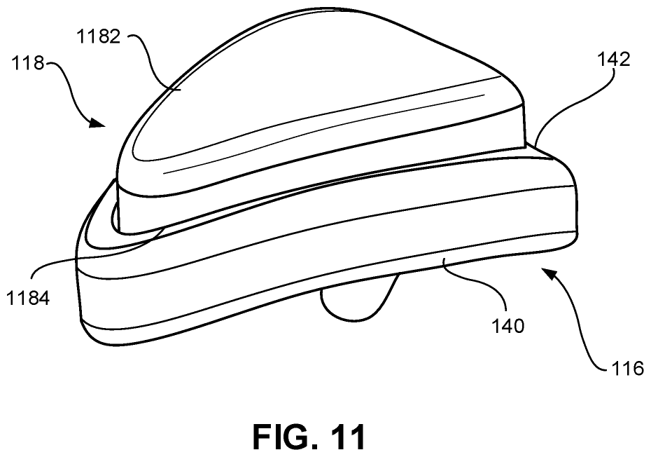

Referring again to FIG. 1, in some implementations, the subtalar joint replacement device 100 includes a bearing insert 118. In some implementations, the bearing insert 118 is coupled to the articular surface 132 of talar component 114, as depicted in FIG. 10, and interacts with an articular surface 142 of the calcaneal component 116. In some implementations, the bearing insert 118 is coupled to the articular surface 142 of the calcaneal component 116, as depicted in FIG. 11, and interacts with the articular surface 142 of the talar component 116. In some implementations, the bearing insert 118 is coupled to the articular surface 142 of the calcaneal component 116 using a press fit or an interference fit. In some implementations, the bearing insert 118 is coupled to the articular surface 142 of the calcaneal component 116 prior to implantation of the calcaneal component 116.

As can be seen in FIGS. 1, 10, and 11, the bearing insert 118 has a convex proximal surface 1182 that interfaces with the talar component 114 of the subtalar joint replacement device 100 and a concave distal surface 1184 that interfaces with the calcaneal component 116 of the subtalar joint replacement device 100. The bearing insert 118 can be formed of or include one or more polymer materials, such as polyethylene, high density polyethylene (HDPE).

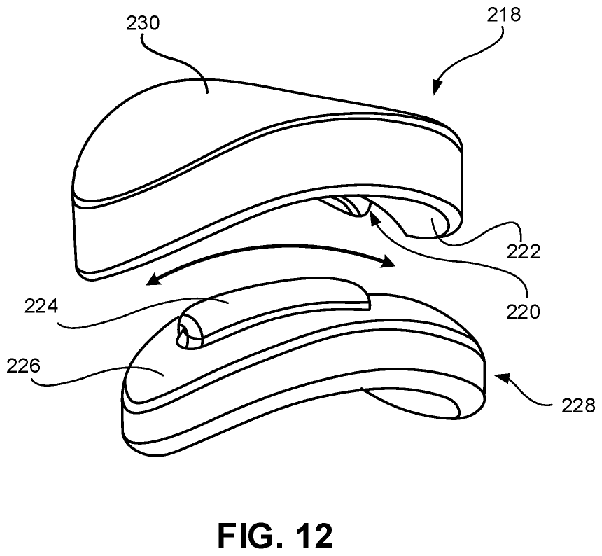

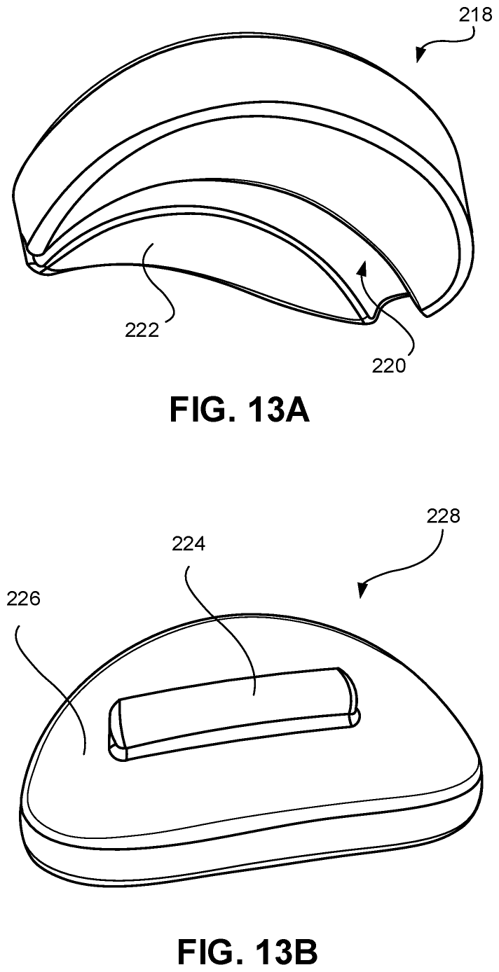

In some implementations, the bearing insert is not fixed to either the talar component 114 or the calcaneal component 116, and is instead free to move independently between the talar component 114 and the calcaneal component 116. For example, as depicted in FIGS. 12 and 13A-13B, the bearing insert 218 can define a slot 220 extending along a surface of the bearing insert 218 that slidably engages a rail extruded along the articular surface 132, 142 of a talar component 114 or calcaneal component 116 of a subtalar joint replacement device 100.

For example, as depicted in FIGS. 12 and 13A-13B, the bearing insert 218 defines a slot 220 on the concave surface 222 of the bearing insert 218 that is configured to interface with a rail 224 on the articular surface 226 of a calcaneal component 228. The slot 220 is designed to allow the bearing insert 218 to slide along the rail 224 on the calcaneal component 228. In some embodiments, the rail 224 can be provided on the articular surface of a talar component of the device 100, and the slot 220 can be provided on the convex surface 230 of the bearing insert 218, allowing the bearing surface 218 to move relative to the talar component along the rail. In some implementations, the rail 224 is extruded onto the articular surface of the talar component or the calcaneal component (or both).

In some implementations, the thickness of the bearing insert 118 can be non-uniform in order to correct one or more deformities of the subtalar joint 102, such as flatfoot and hindfoot deformities. For example, in cases in which the subtalar joint 102 does not present any deformities, a bearing insert with a uniform thickness (e.g., bearing surface 118 of FIGS. 1, 10, and 11) can be used in the subtalar joint replacement device 100. However, if the subtalar joint 102 has one or more deformities, the thickness of the bearing surface 118 can be non-uniform (variable) to correct the joint deformities.

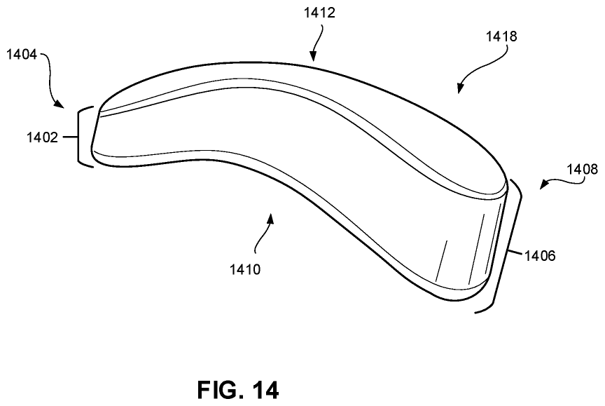

For example, FIG. 14 depicts a bearing insert 1418 with a non-uniform thickness that can be used to correct deformities and improve alignment across the subtalar joint 102. As can be seen in FIG. 14, the bearing insert 1418 has a first thickness 1402 at a medial side 1404 of the bearing insert 1418 and a second, larger thickness 1406 at a lateral side 1408 of the bearing insert 1418, with the thickness of the bearing insert 1418 gradually increasing from the medial side 1404 to the lateral side 1408. The thickness of the bearing insert 1418 can also vary from the posterior side 1410 to the anterior side 1412 of the bearing insert 1418.

By varying the thickness of the bearing insert 1418 in one or more directions, multiplanar deformities within the subtalar joint 102 can be corrected, and alignment across the subtalar joint 102 can be improved. For example, by increasing the thickness of the bearing insert 1418 in the lateral and anterior directions, the alignment of a subtalar joint 102 with valgus hindfoot deformity can be improved.

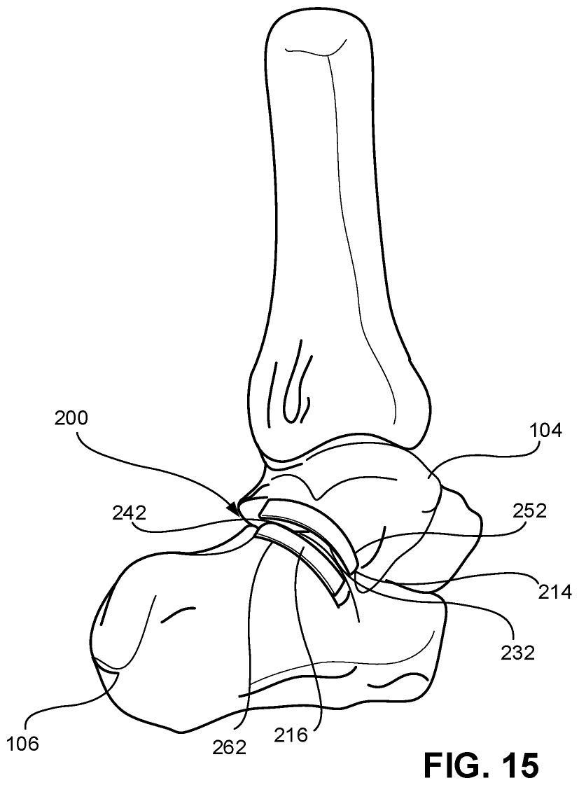

FIG. 15 depicts another example subtalar joint replacement device 200 implanted within the subtalar joint 102 between the talus bone 104 and calcaneus bone 106. As depicted in FIG. 14, the subtalar joint replacement device 200 includes a talar component 214 configured to be attached to the talus bone 104 and a calcaneal component 216 configured to be attached to the calcaneus bone 106. The talar component 214 and the calcaneal component 216 of the subtalar joint replacement device 200 each consist of a monoblock construction, which eliminates the need for a separate bearing component (such as bearing component 118 of FIG. 1). As a result, the talar component 214 and the calcaneal component 216 of the subtalar joint replacement device 200 are configured to directly interface with one another when the subtalar joint replacement device 200 is implanted in the subtalar joint 102. For example, the articular surface 232 of the talar component 214 of the subtalar joint replacement device 200 is configured to contact the articular surface 242 of the calcaneal component 216.

In some implementations, both the talar component 214 and the calcaneal component 216 are composed entirely of a polymer material, such as polyethylene, high density polyethylene (HDPE). In some implementations, the talar component 214 and the calcaneal component 216 of the subtalar joint replacement device 200 each include a polymeric articular surface 232, 242 and one or more inset metal fixation elements (not shown). For example, in some implementations, the talar component 214 and the calcaneal component 216 of the subtalar joint replacement device 200 each include an articular surface 232, 242 composed of high density polyethylene (HDPE) and one or more titanium fixation elements that extend from a fixation surface 252, 262 opposite the respective articular surface 232, 242.

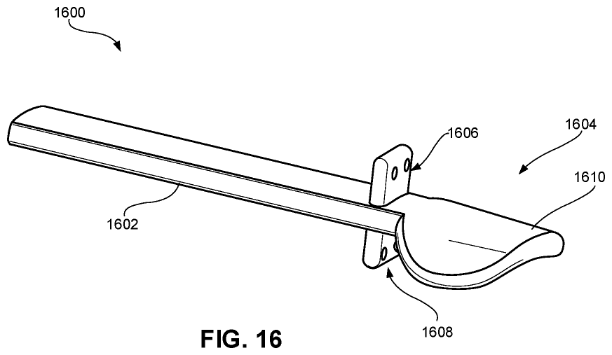

FIGS. 16-20 depict instruments used to implant the subtalar joint replacement device 100.

For example, FIG. 16 depicts an alignment tool 1600 used for installing the subtalar joint replacement device 100. The alignment tool 1600 includes a handle 1602 and a tool end 1604. The tool end 1604 of the alignment tool 1600 is configured to be inserted into the subtalar joint 102 and allows a physician to visualize the alignment of the subtalar joint 102, as well as prepare the talus bone 104 and calcaneus bone 106 for insertion of cutting guides. For example, the tool end 1604 of the alignment tool 1600 includes two sets of holes 1606, 1608 that can be used to guide the insertion of surgical pins into the talus bone 104 and calcaneus bone 106, respectively. As will be described in further detail herein, the surgical pins inserted into the talus and calcaneus bones 104, 106 using the alignment tool 1600 can be used to attach cutting guides to the talus bone 104 and calcaneus bone 106 in a precise position.

In some implementations, the tool end 1604 of the alignment tool 1600 is composed primarily of radiolucent material and includes a set of radiolucent markers that can be used to assess the orientation and size of the subtalar joint 102. For example, radiolucent markers can be arranged on the tool end 1604 of the alignment tool 1600 to indicate the medial-lateral and anterior-posterior edge of the corresponding implant component, which can be used to assess coverage of the corresponding implant 100 within the subtalar joint. In some implementations, the tool end 1604 of the alignment tool 1600 includes an insertion element 1610 that is shaped to conform to the surface of a cone section, which closely matches the native anatomy of the articular surface of the talus bone 104 and calcaneus bone 106, and therefore provides a good reference for intended implant size, position and orientation. By utilizing an alignment tool end 1604 with an insertion element 1610 that has a shape that closely matches the native anatomy the articular surfaces of the talus bone 104 and calcaneus bones 106, the optimal implant size, position, and orientation can be accurately assessed prior to implantation of the subtalar joint replacement device 100.

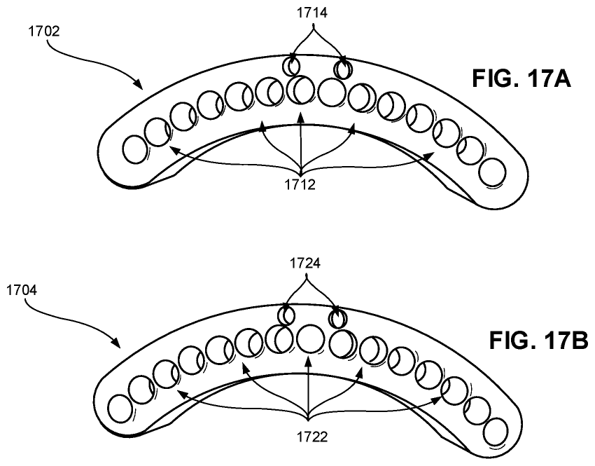

FIG. 17A and FIG. 17B depict a set of cutting guides 1702, 1704 that can be used to form a guided resection the talus bone 104. As depicted in FIG. 17A, the first cutting guide 1702 includes a series of openings 1712 aligned across the length of the cutting guide 1702. In addition, the first cutting guide 1702 includes a pair of holes 1714 near the proximal side of the cutting guide 1702 that can be used to attach the cutting guide 1702 to the talus bone 104. For example, as previously discussed, the alignment tool 1600 can be used to insert a pair of surgical pins into the talus bone 104, and the cutting guide 1702 can then be attached to the talus bone 104 by positioning the cutting guide 1702 against the talus bone 104 such that the surficial pins are inserted through the pair of holes 1714 on the proximal side of the cutting guide 1702. Once the cutting guide 1702 is attached to the talus bone 104, a drilling tool can inserted into each of the openings in the series of openings 1712 in the cutting tool 1702 to form a series of holes through the talus bone 104.

As can be seen in FIGS. 17A and 17B, the second cutting tool 1704 includes a series of openings 1722 aligned across the length of the cutting guide 1704 that are offset from the series of openings 1712 aligned across the length of the first cutting guide 1702. As a result, the second cutting guide 1704 can be used together with the first cutting guide 1702 to form a completely resected surface on the talus bone 104. For example, after drilling a first series of holes in the talus bone 104 using the first cutting guide 1702, the first cutting guide 1702 is removed from the talus bone 104 and the second cutting guide 1704 is attached to the talus bone 104 by positioning the cutting guide 1702 against the talus bone 104 such that the surgical pins are inserted through a pair of holes 1724 near the proximal edge of the second cutting guide 1704. Once the second cutting guide 1704 is attached to the talus bone 104, a drilling tool can inserted into each of the openings 1722 in the cutting tool 1704 to form a second series of holes through the talus bone 104 that are offset from the set of holes created using the first cutting guide 1702. As a result of the offset between the series of openings 1712, 1722 in the first and second cutting guides 1702, 1704, a continuous resected fixation surface is formed on the articular surface of the talus bone 104. The resected fixation surface formed on the talus bone 104 using the cutting guides 1702, 1704 can be used to attach the talar component 114 of the subtalar joint replacement device 100 to the talus bone 104.

In some implementations, as depicted in FIGS. 17A and 17B, the cutting guides 1702, 1704 are shaped to form a fixation surface on the talus bone 104 that complements the fixation surface 130 of the talar component 114 of the subtalar joint replacement device 100. For example, if the talar component 114 has a fixation surface 130 that conforms to the surface of a cone section, the cutting guides 1702, 1704 can be configured to form a resected surface on the talus bone 104 that also conforms to the surface of a cone section.

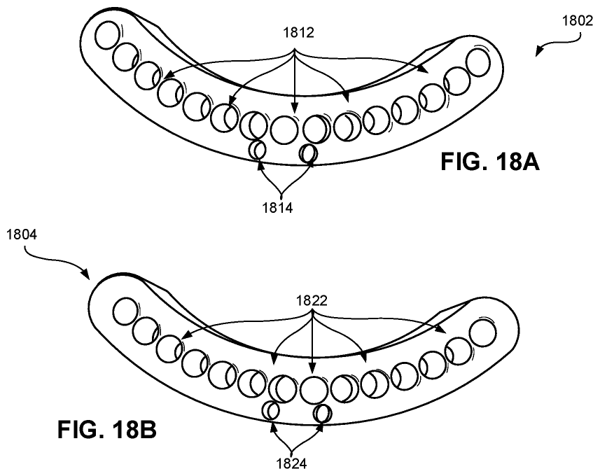

FIGS. 18A and 18B depict a set of cutting guides 1802, 1804 that can be used to form a resected fixation surface on the calcaneus bone 106. As depicted in FIG. 18A, the first cutting guide 1802 includes a series of openings 1812 aligned across the length of the cutting guide 1802. The first cutting guide 1802 also includes a pair of holes 1814 near the distal side of the cutting guide 1802 that can be used to attach the cutting guide 1802 to the calcaneus bone 106. For example, as previously discussed, the alignment tool 1600 can be used to insert a pair of surgical pins into the calcaneus bone 106, and the cutting guide 1802 can then be attached to the calcaneus bone 106 by positioning the cutting guide 1802 against the calcaneus bone 106 such that the surgical pins are inserted through the pair of holes 1814 on the distal side of the cutting guide 1802. Once the cutting guide 1802 is attached to the calcaneus bone 106, a drilling tool can inserted into each of the openings in the series of openings 1812 in the cutting tool 1702 to form a series of holes through the calcaneus bone 106.

As can be seen in FIGS. 18A and 18B, the second cutting tool 1802 includes a series of openings 1822 that are aligned across the length of the second cutting guide 1804. The openings 1822 in the second cutting guide 1804 are offset from the series of openings 1812 of the first cutting guide 1802. As a result, the second cutting guide 1804 can be used together with the first cutting guide 1802 to form a resected surface on the calcaneus bone 106. For example, after drilling a first series of holes in the calcaneus bone 106 using the first cutting guide 1802, the first cutting guide 1802 is removed from the calcaneus bone 106 and the second cutting guide 1804 is attached to the calcaneus bone 106 by inserting surgical pins in the calcaneus bone 106 through the pair of holes 1824 on the distal side of the second cutting guide 1804. Once the second cutting guide 1804 is attached to the calcaneus bone 106, a drilling tool can inserted into each of the openings 1822 in the cutting tool 1804 to form a second series of holes through the calcaneus bone 106 that are offset from the set of holes formed using the first cutting guide 1802. As a result of the offset between the series of openings 1812, 1822 in the first and second cutting guides 1802, 1804, a continuous resected surface is formed on the calcaneus bone 106 and forms a fixation surface for the calcaneal component 116 of the subtalar joint replacement device 100.

Similar to the talar cutting guides 1702, 1704, the calcaneal cutting guides 1802, 1804 can be shaped to form a fixation surface on the calcaneus bone 106 that complements the fixation surface 140 of the calcaneal component 116 of the subtalar joint replacement device 100. For example, if the calcaneal component 116 has a fixation surface 140 that conforms to the surface of a cone section, the cutting guides 1802, 1804 can be configured to form a resected surface on the calcaneus bone 106 that also conforms to the surface of a cone section.

In some implementations, the alignment tool 1600, talar cutting guides 1702, 1704, and calcaneal guides 1802, 1804 used to form resected fixation surfaces on the talus bone 104 and calcaneus bone 106, respectively, are standard cutting guides that can be used on all patients. In some implementations, the cutting guides are patient-specific cutting guides that are designed to more closely match the bony shapes of the talus bone 104 and calcaneus bone 106 of the particular patient.

For example, a CT scan of the subtalar joint 102 of the patient can be used to reconstruct the 3-dimensional geometries of the bones 104, 106 of the subtalar joint 102 in a neutral position. Based on the 3D reconstruction, the optimal size, shape and position for both the talar component 114 and calcaneal component 116 of the subtalar joint replacement device 100 are determined and a pair of patient-specific talar cutting guides and a pair of patient-specific calcaneal cutting guides can be formed with a surface that matches the posterolateral surface of the talus bone 104 and calcaneus bone 106, respectively, in the reconstruction.

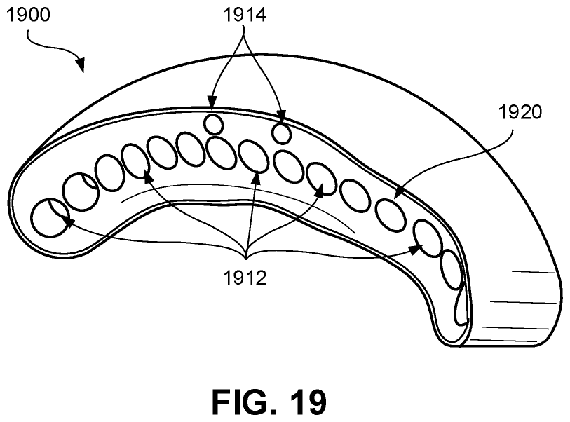

FIG. 19 depicts a patient-specific talar cutting guide 1900. Similar to cutting guides 1702, 1704 depicted in FIGS. 17A and 17B, the patient-specific talar cutting guide 1900 includes a series of openings 1912 aligned across the length of the cutting guide 1900 and a pair of holes 1914 near the proximal edge of the cutting guide 1900 that can be used to attach the cutting guide 1900 to the talus bone 104 via surgical pins. As depicted in FIG. 19, the back surface 1920 of the patient-specific cutting guide 1900 is contoured to match the bony shapes of surface of the talus bone 104 of the patient. For example, the back surface 1920 of the patient-specific cutting guide 1900 can be configured to match the posterolateral surface of the talus bone 104 of the patient. As discussed in reference to FIGS. 17-18, each of the talar cutting guides includes a set of openings that are offset from one another, which allows for a resected surface to be formed on the talus bone 104 using each of the patient-specific talar cutting guides in sequence. In addition, a pair of patient-specific calcaneal cutting guides (not shown) can be formed based on the 3D reconstruction of the calcaneus bone 106 to include a surface that matches the surface of the calcaneus bone 106. For example, the back surface of the patient-specific calcaneal cutting guides can be configured to match the posterolateral surface of the calcaneus bone 106 of the patient. Like the patient-specific talar cutting guides, each of the calcaneal cutting guides can include a set of openings that are offset from one another, which allows for a resected surface to be formed on the calcaneus bone 106 when each of the patient-specific calcaneal cutting guides are used in sequence.

In some implementation, by forming patient-specific talar cutting guides and patient-specific calcaneal cutting guides (such as cutting guide 1900 of FIG. 19), the surfaces of the talus bone 104 and the surface of the calcaneus bone 106 can be resected without the use of an alignment tool (e.g., alignment tool 1600 of FIG. 16). For example, because the surface of each of the patient-specific cutting guides is configured to closely correspond to the native surface of the respective talus and calcaneus bones 104, 106, the patient-specific cutting guides can be properly positioned against the respective bones 104, 106 without the need for a separate alignment tool.

Further, while the patient-specific cutting guides 1900 depicted in FIG. 19 includes a set of holes 1914 for pinning the surgical guide 1900 to the respective bone, in some implementations, patient-specific guides do not require pin holes to attach the guides to the bones 104, 106 of the patient. For example, in some implementations, the surface 1920 of the patient-specific guide 1900 so closely matches the native anatomy of the talus bone 104 (e.g., the postlateral surface of the talus bone 104) that positioning the surface 1920 of patient-specific guide 1900 against the corresponding surface of the talus bone 104 temporarily attaches the guide 1900 to the bone 104 in the correct position.

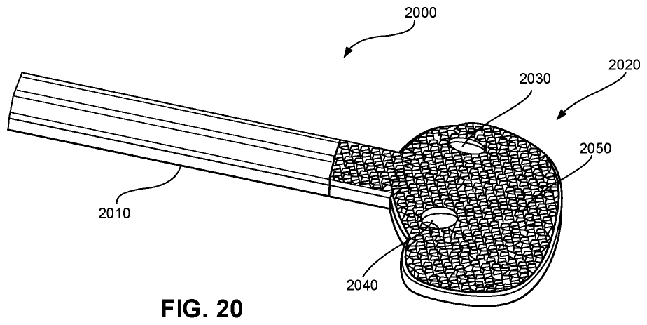

FIG. 20 depicts a surface prep tool 2000 for preparing the resected surface of the talus bone 104 and the calcaneus bone 106 for implantation of a subtalar joint replacement device. As can be seen in FIG. 20, the surface prep tool 2000 includes a handle 2010 and a tool end 2020. The tool end 2020 of the surface prep tool 2000 can be used to prepare the resected surface of the talus bone 104 and/or the calcaneus bone 106 for implantation of the subtalar joint replacement device 100. For example, the tool end 2020 of the surface prep tool 2000 include a pair of openings 2030, 2040 that can be used to guide a hole punch or drill to create a set of holes in the resected surface formed on the talus bone 104 and/or calcaneal surface 106, which are then used to secure the replacement device 100 to the respective bones 104, 106 (e.g., via a press fit with pins, pegs, posts, keels, etc. on each of the components 114, 116 of the replacement device 100).

In some implementations, the tool end 2040 of the surface prep tool 2000 has a rough surface 2050 that can be used to smooth the resected surface formed on the talus bone 104 and/or calcaneus bone 106. For example, the surface 2050 of the tool end 2040 of the surface prep tool 2000 can be scrubbed against the resected surface formed on the talus bone 104 and/or on the calcaneus bone 106 using the cutting guides to remove any imperfections from the resected surface on the respective bones.

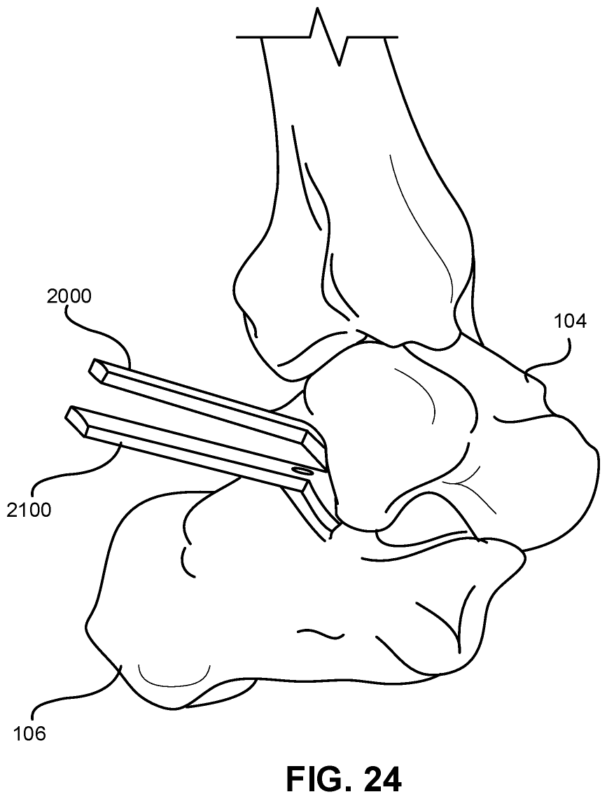

In some implementations, as depicted in FIG. 24, separate surface prep tools 2000, 2100 can be provided for preparing the talus bone 104 and the calcaneus bone 106 for implantation of the replacement device 100. For example, a first surface prep tool 2000 that is sized and shaped to correspond to the resected surface of the talus bone 104 can be used to remove any imperfections from the resected surface of the talus bone 104, and a second surface prep tool 2100 that is sized and shaped to correspond to the resected surface of the calcaneus bone 106 can be used to remove any imperfections from the resected surface of the calcaneus bone 106. In addition, each of the surface prep tools 2000, 2100 can include guides for inserting fixation devices into the respective bones 104, 106.

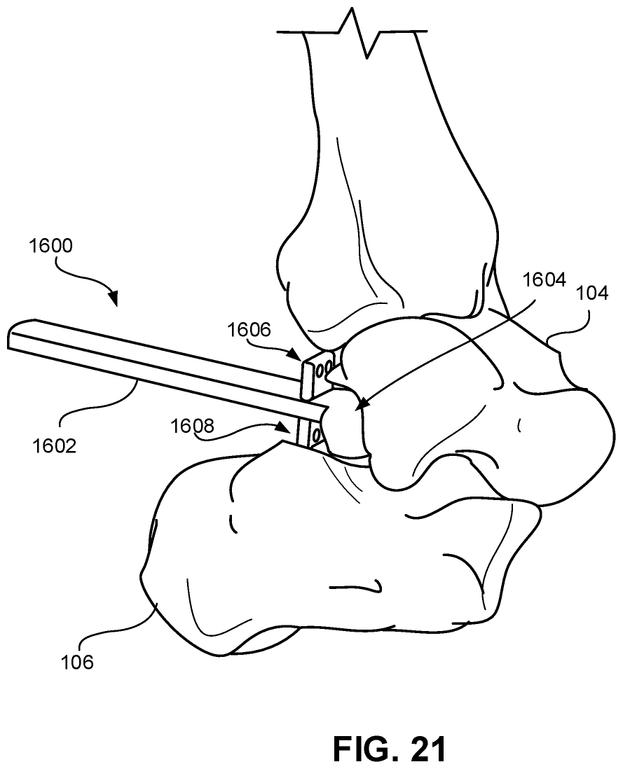

A method of implanting the subtalar joint replacement device 100 will now be described with reference to FIGS. 21-28.

As previously discussed, the subtalar joint replacement device 100 is intended for the replacement, realignment, and resurfacing of the posterior facet of a diseased subtalar joint 102. In some implementations, the procedure for implanting the subtalar joint replacement device 100 is performed through a posterior lateral approach to the hindfoot. For example, in order to implant the subtalar joint replacement device 100 into the subtalar joint 102, a posterior-lateral arthrotomy is performed to provide the physician with access to the subtalar joint 102.

As depicted in FIG. 21, after an arthrotomy is performed to expose the subtalar joint 102, a physician can use the handle 1602 of an alignment tool 1600 to position the tool end 1604 of the alignment tool 1600 within the subtalar joint 102 between the talus bone 104 and the calcaneus bone 106. Once positioned within the subtalar joint 102, the alignment tool 1600 is used to determine the orientation of the subtalar joint and the appropriate size device 100 for implanting in the patient's subtalar joint 102. Under fluoroscopic guidance, orientation of the subtalar joint is confirmed by comparing the shape of the talus bone 104 and calcaneus bone 106 to that of the alignment tool 1600, and the coverage of the alignment tool 1600 within the subtalar joint 102 is used to determine the correct implant 100 size.

As depicted in FIG. 21, when the tool end 1604 of an alignment tool 1600 is positioned within the subtalar joint 102, the first set of holes 1606 is positioned against the talus bone 104 and the second set of holes 1608 is positioned against the calcaneus bone 106. A drill tool is then inserted through each of the sets of holes 1606, 1608 in the alignment tool 1600 to form openings through the talus bone 1604 and calcaneus bone 1606.

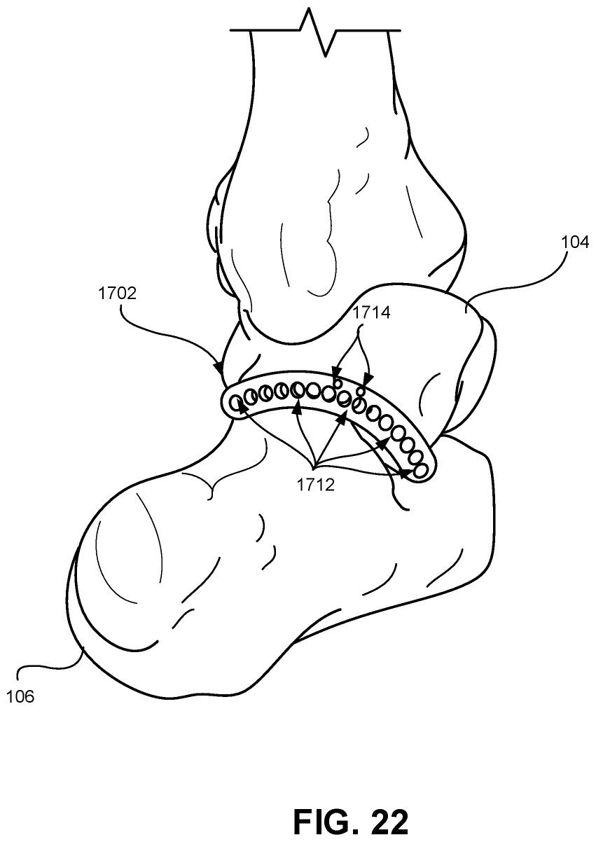

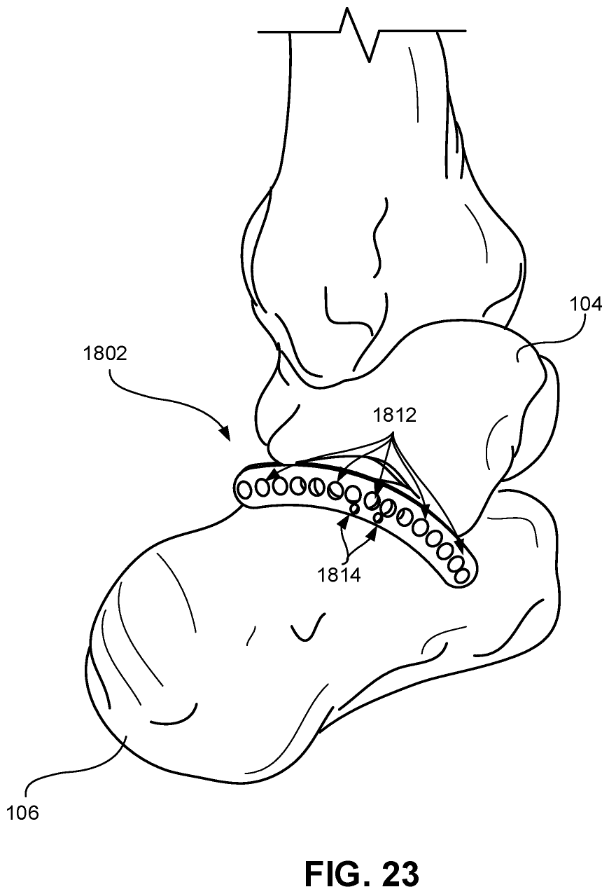

Referring to FIGS. 22 and 23, once the holes are formed in the talus bone 104 and calcaneus bone 106, cutting guides are placed individually over the talus and calcaneus bones 104, 106. In some implementations, surgical pins (not shown) are inserted into each of the openings formed in the talus bone 104 and calcaneus bone 106 and are used to attach the cutting guides to the respective bones 104, 106. In some implementations, the drill bits used to form the holes in the bones 104, 106 are left in one or more of the holes and are used to keep the cutting guides in place while the remaining holes are drilled into the respective bones 104, 106 to form a resected surface.

For example, as shown in FIG. 22, a first cutting guide 1702 is attached to the talus bone 104 by inserting surgical pins through the openings 1714 near the proximal side of the cutting guide 1702 and into the holes formed in the talus bone 104. Once the first cutting guide 1702 is attached to the talus bone 104, a drill tool can be inserted into each of the plurality of openings 1712 in the first cutting guide 1702 to form as first set of holes in the articular surface of the talus bone 104.

Once the first set of holes is drilled into the articular surface of the talus bone 104, the first cutting guide 1702 is removed from the bone 104 and a second cutting guide with openings that are offset from the first cutting guide (e.g., cutting guide 1704 of FIG. 17B) is positioned against the talus bone 104 by inserting the surgical pins through the openings 1724 in the second cutting guide 1704. Once the second cutting guide 1704 is attached to the talus bone 104, a drill tool can be inserted into each of the plurality of openings 1722 in the second cutting guide 1704 to form as second set of holes in the articular surface of the talus bone 104.

As a result of the offset between the series of openings 1712, 1722 in the first and second cutting guides 1702, 1704, use of the first and second cutting guides 1702, 1704 in sequence forms a resected surface on the articular surface of the talus bone 104. As previously discussed, in some implementations, the cutting guides 1702, 1704 are shaped to form a resected surface on the talus bone 104 that complements a fixation surface 130 of the talar component 114 of the subtalar joint replacement device 100 that is being implanted. For example, if the talar component 114 being implanted has a fixation surface 130 that conforms to the surface of a cone section, the cutting guides 1702, 1704 can be configured to form a resected surface on the talus bone 104 that also conforms to the surface of a cone section. In some implementations, the series of openings 1712, 1722 in the first and second talar cutting guides 1702, 1704 form a path that rotates about the same cone axis (e.g., cone axis 502 of FIG. 5) as the fixation surface 130 of the talar component 114.

Once a resected surface has been formed on the talus bone 104, the second cutting guide 1704 is removed from the talus bone 104, and a first cutting guide is attached to the calcaneus bone 106. For example, as shown in FIG. 23, a first cutting guide 1802 is attached to the calcaneus bone 106 by inserting pins through the openings 1814 near the distal end of the cutting guide 1802 and into the holes formed in the calcaneus bone 106 using the alignment tool 1600. Once the first cutting guide 1802 is attached to the calcaneus bone 106, a drill tool can be inserted into each of the plurality of openings 1812 in the first cutting guide 1802 to form as first set of holes in the surface of the calcaneus bone 106.

Once the first set of holes is drilled into the calcaneus bone 106, the first cutting guide 1802 is removed from the calcaneus bone 106 and a second cutting guide with openings that are offset from the first cutting guide (e.g., cutting guide 1804 in FIG. 18B) is positioned against the calcaneus bone 106 by inserting the pins through the openings 1824 near the distal side of the second cutting guide 1804. Once the second cutting guide 1804 is attached to the calcaneus bone 106, a drill tool can be inserted into each of the plurality of openings 1822 in the second cutting guide 1804 to form as second set of holes in the surface of the calcaneus bone 106. Once the second set of holes is formed into the calcaneus bone 106, the second cutting guide 1804 and any pins used to hold the cutting guides 1802, 1804 to the calcaneus bone 106 are removed.

As with the talar cutting guides 1702, 1704, the offset between the series of openings 1812, 1822 in the first and second calcaneal cutting guides 1802, 1804 results in the formation of a continuous resected surface on the articular surface of the calcaneus bone 106 when the first and second cutting guides 1702, 1704 are used in sequence. As previously discussed, in some implementations, use of the cutting guides 1802, 1804 forms a resected surface on the calcaneus bone 106 that complements a fixation surface 140 of the calcaneal component 116 of the subtalar joint replacement device 100. For example, if the calcaneal component 116 being implanted has a fixation surface 140 that conforms to the surface of a cone section, the cutting guides 1802, 1804 can be configured to form a resected surface on the articular surface of the calcaneus bone 106 that also conforms to the surface of a cone section. In some implementations, the series of openings 1812, 1822 in the first and second cutting guides 1802, 1804 form a path that rotates about the same cone axis (e.g., cone axis 602 of FIG. 6) as the fixation surface 140 of the calcaneal component 116.

As depicted in FIGS. 17A, 17B, 18A, and 18B, the cutting guides 1702, 1704, 1802, 1804 each include a curved path of openings 1712, 1722, 1812, 1822 that can be used to create curved cuts in the talar and calcaneus bones 104, 160 through serial drilling through the curved series of openings 1712, 1722, 1812, 1822. By creating curved cuts that mimic native anatomy of the bones 104, 106, the cutting guides 1702, 1704, 1802, 1804 allow for minimized amounts of bony resection and maximize the bone support for fixation of the subtalar joint replacement device 100 by the natively curved joint 102.

In some implementations, the resected surface is formed on the calcaneus bone 106 prior to forming the resected surface on the talus bone 104. In some implementations, the talar cutting guides and the calcaneal cutting guides are patient-specific cutting guides having a surface that conforms to the bony surface of the talus bone 104 or calcaneus bone 106, respectively, of the patient.

In some implementations, a physician selects the talar cutting guides 1702, 1704 and calcaneal cutting guides 1802, 1804 from a series of cutting guides, each with slightly varying configurations to allow for precise adjustments to the resection level, angle, or position.

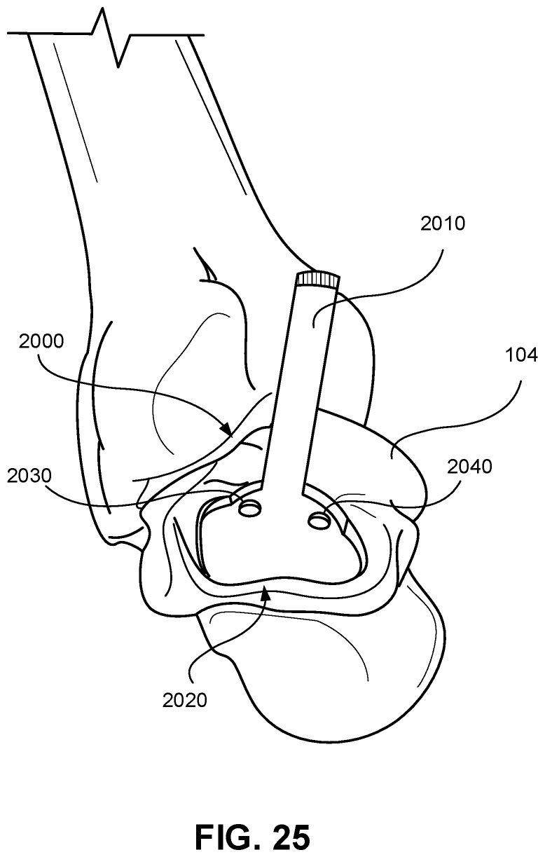

Referring to FIG. 24, once a resected surface has been formed on the talus bone 104 and on the calcaneus bone 106 and the cutting guides 1702, 1704, 1802, 1804 and pins (if any) have been removed, surface prep tools 2000, 2100 can be used to prepare the resected surfaces for fixation of the components 114, 116 of the subtalar joint replacement device 100 to the talus and calcaneus bones 104, 106. For example, as depicted in FIG. 25, the tool end 2020 of the resection device 2000 can be positioned flush with the resected surface of the talus bone 104 and the rough surface 2050 of the tool end 2020 of the resection device 2000 can be brushed against the resected surface of the talus bone 104 to further smooth the resected surface on the talus bone 104. In addition, a drill tool or hole punch can be inserted through the openings 2030, 2040 of the surface prep tool 2000 to form corresponding openings 2035, 2045 in the talus bone 104 when the tool end 2020 of the resection device 2000 is positioned flush with the resected articular surface of the talus bone 104. As will be described in further detail herein, the opening 2035, 2045 formed in the talus bone 104 using the surface prep tool 2000 are configured to couple with fixation elements of the talar component 114 of the subtalar joint replacement device 100 and attach the talar component 114 to the talus bone 104.

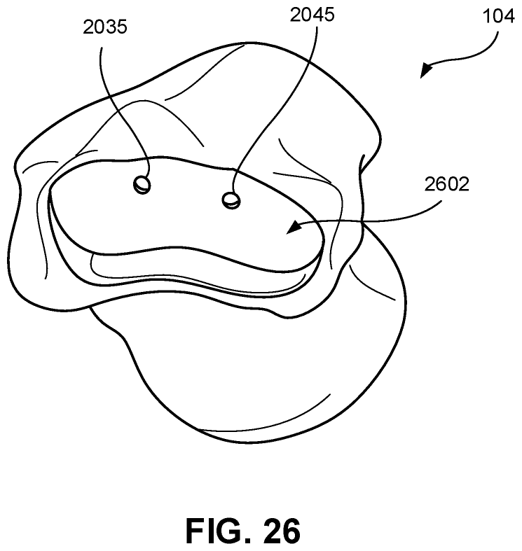

FIG. 26 depicts the talus bone 104 with a smooth, resected surface 2602 and openings 2035, 2045 formed through the talus bone 104 to fix the talar component 114 of the subtalar joint replacement device 100 to the talus bone 104. As previously discussed, in some implementations, the shape of resected surface 2602 formed on the talus bone 104 using the cutting guides 1702, 1704 and surface prep tool 2000 corresponds to the shape of the fixation surface 130 of the talar component 114.

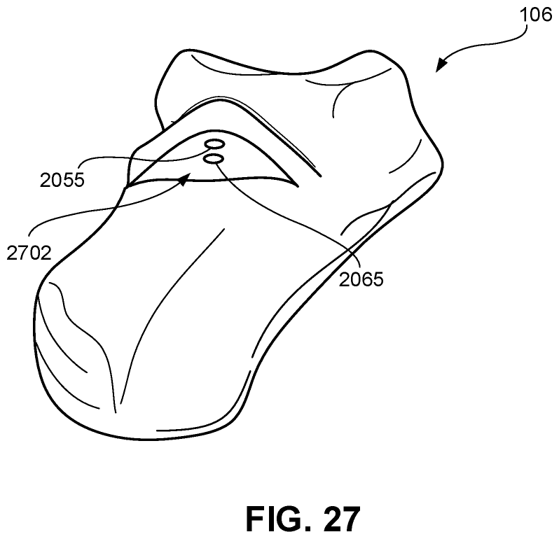

Referring to FIGS. 25 and 27, a tool end of the resection device 2100 (similar to tool end 2020) can be positioned flush with the resected articular surface of the calcaneus bone 106. A rough surface of the tool end of the resection device 2100 can be brushed against the resected surface of the calcaneus bone 106 to further smooth the resected surface. In addition, a drill tool or hole punch can be inserted through openings (e.g., openings 2030, 2040) of the surface prep tool 2100 to form corresponding openings 2055, 2065 in the calcaneus bone 106 when the tool end of the resection device 2100 is positioned flush with the resected articular surface of the calcaneus bone 106. As will be described in further detail herein, the openings 2055, 2065 formed in the calcaneus bone 106 using the surface prep tool 2100 are configured to couple with a fixation elements of the calcaneal component 116 of the subtalar joint replacement device 100 and attach the calcaneal component 116 to the calcaneus bone 106.

FIG. 27 depicts the calcaneus bone 106 with a smooth resected surface 2702 and openings 2055, 2065 formed through the calcaneus bone 106 to fix the calcaneal component 116 of the subtalar joint replacement device 100 to the calcaneus bone 106. As previously discussed, in some implementations, the shape of the resected surface 2702 formed on the calcaneus bone 106 using the cutting guides 1802, 1804 and surface prep tool 2100 corresponds to the shape of the fixation surface 140 of the calcaneal component 116.

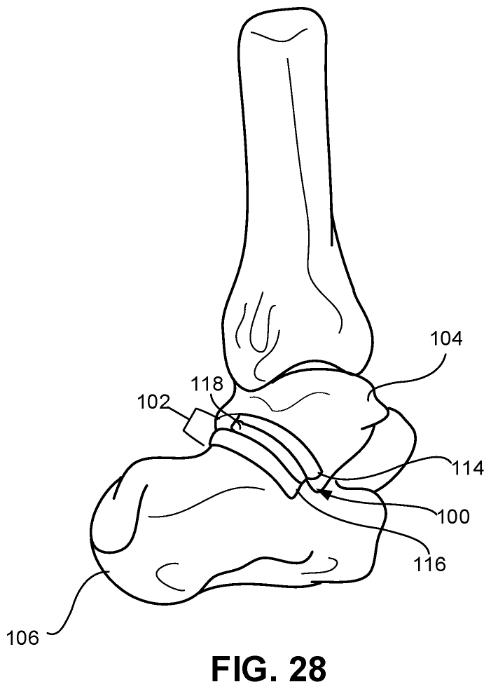

Referring to FIG. 28, once the resected surfaces 2602, 2702 have been formed on the articular surfaces of the talus bone 104 and calcaneus bone 106 and openings 2035, 2045, 2055, 2065 for attaching the subtalar joint replacement device 100 have been formed in each bone 104, 106, the talar component 114 of the subtalar joint replacement device 100 is attached to the talus bone 104 and the calcaneal component 116 of the subtalar joint replacement device 100 is attached to the calcaneus bone 106. In some implementations, each of the components 114, 116 of the subtalar joint replacement device 100 are impacted into place by inserting the fixation devices 134, 136, 144, 146 of the talar and calcaneal components into the openings 2035, 2045, 2055, 2065 formed into the bones 104, 106.

For example, fixation devices extending from the fixation surface 130 of the talar component 114 (e.g., fixation devices 134, 136) can be inserted and press fit into the openings 2035, 2045 formed into the talus bone 104 (e.g., using an impacting tool). As can be seen in FIG. 28, when the fixation devices of the talar component 114 are fully inserted into the openings 2035, 2045 formed into the talus bone 104, the fixation surface 130 of the talar component 114 is flush with the resected surface 2602 on the talus bone 104.

Similarly, fixation devices extending from the fixation surface 140 of the calcaneal component 116 (e.g., fixation devices 144, 146) can be inserted and press fit into the openings 2055, 2065 formed into the calcaneus bone 106 (e.g., using an impacting tool). As can be seen in FIG. 28, when the fixation devices of the calcaneal component 116 are fully inserted into the openings 2055, 2056 formed into the calcaneus bone 106, the fixation surface 140 of the calcaneal component 116 is flush with the resected surface 2702 on the calcaneus bone 106.

A number of embodiments have been described. Nevertheless, it will be understood that various modifications may be made without departing from the spirit and scope of the disclosure. Accordingly, other embodiments are within the scope of the following claims.

What is claimed is:

1. A subtalar joint replacement device comprising:
a talar component comprising:
a talar fixation surface;
a talar articular surface opposite the talar fixation surface, the talar articular surface having a first shape; and
one or more fixation devices extending from the talar fixation surface;
a calcaneal component comprising:
a calcaneal fixation surface;
a calcaneal articular surface opposite the calcaneal fixation surface, the calcaneal articular surface having a second shape that interfaces with the first shape of the talar articular surface to generate constraint across a subtalar joint; and
one or more fixation devices extending from the calcaneal fixation surface.

2. The subtalar joint replacement device of claim 1, wherein:
the first shape of the talar articular surface conforms to a surface of a portion of a first torus; and
the second shape of the calcaneal articular surface conforms to a surface of a portion of a second torus.

3. The subtalar joint replacement device of claim 2, wherein:
the first torus has a first articular radius; and
the second torus has a second articular radius different from the first articular radius.

4. The subtalar joint replacement device of claim 1, wherein:
the talar fixation surface is shaped to conform to native anatomy of an articular surface a talus bone; and
the calcaneal fixation surface is shaped to conform to native anatomy of an articular surface a calcaneus bone.

5. The subtalar joint replacement device of claim 4, wherein:
the talar fixation surface is shaped to conform to a surface of a section of a first cone; and
the calcaneal fixation surface is shaped to conform to a surface of a section of a second cone.

6. The subtalar joint replacement device of claim 1, wherein at least one of the talar component and the calcaneal component has a non-uniform thickness.

7. The subtalar joint replacement device of claim 6, wherein at least one of the talar component and the calcaneal component has a thickness that increases in a lateral direction and in an anterior direction.

8. The subtalar joint replacement device of claim 1, further comprising a bearing insert configured to be positioned between the talar component and the calcaneal component.

9. The subtalar joint replacement device of claim 8, wherein the bearing insert is affixed to the talar articular surface or the calcaneal articular surface.

10. The subtalar joint replacement device of claim 8, further comprising:
a rail formed on the talar articular surface and the calcaneal articular surface; and
a slot formed on at least one surface of the bearing insert, the slot configured to slidably engage the rail.

11. A system comprising:
a subtalar joint replacement device, the subtalar joint replacement device comprising:
a talar component; and
a calcaneal component; and
a set of talar cutting guides;
a set of calcaneal cutting guides; and
a surface prep tool.

12. The system of claim 11, wherein:
the set of talar cutting guides comprises:
a first talar cutting guide with a first set of openings extending along a curved length of the first talar cutting guide; and
a second talar cutting guide with a second set of openings extending along a curved length of the second talar cutting guide, the second set of openings being offset from the first set of openings; and
the set of calcaneal cutting guides comprises:
a first calcaneal cutting guide with a third set of openings extending along a curved length of the first calcaneal cutting guide; and
a second calcaneal cutting guide with a fourth set of openings extending along a curved length of the second calcaneal cutting guide, the fourth set of openings being offset from the third set of openings.

13. The system of claim 12, further comprising an alignment tool configured to indicate an orientation of a subtalar joint and a size of the subtalar joint.

14. The system of claim 13, wherein the alignment tool comprises:
a handle; and
a tool end coupled to the handle, the tool end comprising two or more guide holes.

15. The system of claim 14, wherein the tool end of the alignment tool comprises one or more radiopaque markers.

16. The system of claim 11, wherein:
the set of talar cutting guides comprises a first and second patient-specific talar cutting guide each having a surface that conforms to a surface of a talar bone of a patient;
and the set of calcaneal cutting guides comprises a first and second patient-specific calcaneal cutting guides each having a surface that conforms to a surface of a calcaneal bone of the patient.

17. The system of claim 11, wherein the surface prep tool comprises:
a handle; and
a tool end coupled to the handle, the tool end comprising:
one or more holes; and
a roughened surface.

18. The system of claim 11, further comprising a bearing insert configured to be positioned between the talar component and the calcaneal component.

19. The system of claim 11, wherein
the talar component comprises:
a talar fixation surface;
a talar articular surface opposite the talar fixation surface, the talar articular surface having a first shape; and
the calcaneal component comprises:
a calcaneal fixation surface; and
a calcaneal articular surface opposite the calcaneal fixation surface, the calcaneal articular surface having a second shape that interfaces with the first shape of the talar articular surface to generate constraint across a subtalar joint.

20. A method comprising:
determining an orientation and a size of a subtalar joint of a patient;
forming a curved resected surface on an articular surface of a talus bone of the patient using a first pair of cutting guides, the curved resected surface having a shape that conforms to native anatomy of the articular surface the talus bone;
forming a curved resected surface on an articular surface of a calcaneus bone of the patient using a first second of cutting guides, the curved resected surface having a shape that conforms to native anatomy of the articular surface the calcaneus bone;
coupling a talar component of a subtalar joint replacement device to the talus bone, the talar component having a fixation surface corresponding to the shape of the curved resected surface on the articular surface of the talus bone; and
coupling a calcaneal component of the subtalar joint replacement device to the calcaneus bone, the calcaneal component having a fixation surface corresponding to the shape of the curved resected surface on the articular surface of the calcaneus bone.
